(19) United States
(12) Patent Application Publication
(10) Pub. No.: US 2014/0228662 A1
(43) Pub. Date: Aug. 14, 2014

(54) BIO-ELECTRODE DEVICE, BIO-MEASUREMENT DEVICE, AND METHOD FOR IMPLEMENTING BIO-ELECTRODE DEVICE

(71) Applicant: SAMSUNG ELECTRONICS CO., LTD., Suwon-si (KR)

(72) Inventors: Sang Yun PARK, Hwaseong-si (KR); Byung Hoon KO, Hwaseong-si (KR)

(73) Assignee: SAMSUNG ELECTRONICS CO., LTD., Suwon-si (KR)

(21) Appl. No.: 14/160,858

(22) Filed: Jan. 22, 2014

(30) Foreign Application Priority Data
Feb. 12, 2013 (KR) 10-2013-0014825

(51) Int. Cl. A61B 5/04; A61B 5/00

(52) U.S. Cl. CPC A61B 5/04; A61B 5/6801
USPC 600/372

(57) ABSTRACT

A bio-electrode device, bio-measurement device, and a method for implementing a bio-electrode device are provided. Information, such as usage information or patient information, may be maintained in a bio-electrode device, and the information maintained may be transferred to a bio-measurement device in response to the bio-electrode device being coupled to the bio-measurement device.

### CROSS-REFERENCE TO RELATED APPLICATION

This application claims the benefit under 35 U.S.C. §119(a) of Korean Patent Application No. 10-2013-0014825 filed on Feb. 12, 2013, in the Korean Intellectual Property Office, the entire disclosure of which is incorporated herein by reference for all purposes.

### BACKGROUND

1. Field

The following description relates to a bio-electrode device, a bio-measurement device that uses a bio-electrode device, and a method for implementing a bio-electrode device.

2. Description of Related Art

A bio-measurement device may be used to measure a bio-signal or bio-information related to a user by fastening a bio-electrode device, using disposable supplies, to the user. Such a bio-measurement device may require several forms of information and settings for enhancing a measurement precision, and the like, in order to obtain sufficient information so that the information may be combined to generate high-quality data. Here, the information may refer to characteristic data associated with the user and their physiology or personal attributes, or property data associated with the bio-electrode device.

In general, inputting information and settings for a bio-measurement device is performed via an input device of the bio-measurement device, for example, a keyboard, a touch panel, and the like. Accordingly, producing a compact bio-measurement device has faced problems due to difficulties that have arisen in integrating input devices into the bio-measurement device as such input devices are added, and another problem in that information needs to be inputted again when the bio-electrode device is replaced.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a bio-electrode device includes a bio-electrode, and an electrode pad including a plurality of terminals, wherein a first terminal from among the plurality of terminals is connected to the bio-electrode, and the electrode pad maintains information using a second terminal not connected to the bio-electrode, from among the plurality of terminals.

The information may be maintained based on whether at least a portion of the second terminal is connected to a ground.

The information may be maintained based on whether at least a portion of the second terminal is connected to another terminal of the electrode pad.

The bio-electrode device may further include a dot matrix comprising at least one of a conductive member and an insulating member.

The dot matrix may be configured to maintain information using an array of the conductive member and the insulating member.

The bio-electrode device may further include an optical dot matrix comprising at least one of a transparent member and a non-transparent member.

The optical dot matrix may be configured to maintain information using an array of the transparent member and the non-transparent member.

In another general aspect, a bio-measurement device includes a bio-electrode device, including a bio-electrode, and an electrode pad including a plurality of terminals, wherein a first terminal from among the plurality of terminals is connected to the bio-electrode, and the electrode pad is configured to maintain information using a second terminal not connected to the bio-electrode, from among the plurality of terminals, and a bio-measurement processor operatively connected to the bio-electrode device and configured to read the information from the bio-electrode device.

The bio-measurement processor may be configured to read information from the bio-electrode based on whether at least a portion of the second terminal, from among the plurality of terminals, is connected to a ground.

The bio-measurement processor may be configured to read information from the bio-electrode based on whether at least a portion of the second terminal, from among the plurality of terminals, is connected to another terminal of the electrode pad.

The bio-electrode device may include a dot matrix comprising an array of a conductive member and an insulating member, and the bio-measurement processor may be configured to read information from the bio-electrode based on the array of the conductive member and the insulating member.

The bio-electrode device may include an optical dot matrix including an array of a transparent member and a non-transparent member, and the bio-measurement processor may be configured to read information from the bio-electrode based on the array of the transparent member and the non-transparent member.

The bio-electrode device may be configured such that a first terminal of the plurality of terminals is connected to the bio-electrode, and at least a portion of a second terminal, not connected to the bio-electrode, from among the plurality of terminals, is connected to a ground.

In another general aspect, a bio-electrode device includes a bio-electrode, and an electrode pad including a plurality of terminals, wherein a first terminal of the plurality of terminals is connected to the bio-electrode, and at least a portion of at least one second terminal not connected to the bio-electrode, from among the plurality of terminals, is connected to another terminal of the electrode pad.

In another general aspect, a method for implementing a bio-electrode device includes connecting a first terminal of a plurality of terminals of an electrode pad with a bio-electrode; and maintaining information using a second terminal not connected to the bio-electrode, from among the plurality of terminals of the electrode pad.

The method may further include extracting the information based on whether at least a portion of the second terminal is connected to a ground.

The method may further include extracting the information based on whether at least a portion of the second terminal is connected to another terminal of the electrode pad.

The method may further include connecting a terminal of a plurality of terminals of an electrode pad with a bio-electrode, setting a dot matrix including a conductive member and an insulating member to be a portion of an area of the electrode pad, and maintaining information, by the dot matrix, using an array of the conductive member and the insulating member.

The method may further include connecting a terminal of a plurality of terminals of an electrode pad with a bio-electrode, setting an optical dot matrix including a transparent member and a non-transparent member to be a portion of an area of the electrode pad, and maintaining information, by the optical dot matrix, using an array of the transparent member and the non-transparent member.

In another aspect, a method of producing a customized bio-measurement device includes receiving a selection of configuration information for a bio-electrode device, the bio-electrode device comprising a bio-electrode and first terminal connected to the bio-electrode and a second terminal not connected to the bio-electrode, configuring a portion of the bio-electrode device to store the configuration information, and forming an operative connection between the bio-electrode device and a bio-measurement processor, wherein the operative connection allows the bio-measurement processor to read the configuration information.

The method may provide that the portion of the bio-electrode device is the second terminal and the bio-electrode device is configured to store the configuration information by connecting at least a portion of a second terminal to a ground.

The method may provide that the portion of the bio-electrode device is the second terminal and the bio-electrode device is configured to store the configuration information by connecting at least a portion of a second terminal to another terminal of the electrode pad.

The method may provide that the portion of the bio-electrode device is configured to store the configuration information by using a dot matrix, using an array of a conductive member and an insulating member.

The method may provide that the portion of the bio-electrode device is configured to store the configuration information by using an optical dot matrix, using an array of a transparent member and a non-transparent member

Other features and aspects will be apparent from the following detailed description, the drawings, and the claims.

### BRIEF DESCRIPTION OF THE DRAWINGS

FIG. 1 is a diagram illustrating an example of fastening a bio-electrode device to a bio-measurement device.

FIG. 2 is a diagram illustrating an example of a detailed configuration of a bio-electrode device that maintains information.

FIG. 3 is a diagram illustrating another example of a detailed configuration of a bio-electrode device that maintains information.

FIG. 4 is a diagram illustrating an example of detailed configuration of a bio-electrode device including a matrix that maintains information.

FIG. 5 is a diagram illustrating another example of a detailed configuration of a bio-electrode device including a matrix that maintains information.

FIG. 6 is a flowchart illustrating an example of a method for implementing a bio-electrode device.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

A bio-electrode may refer to a type of sensor for detecting information, such as biological information associated with a body. For example, a bio-electrode occurs in a living body in a form that measures an electrical signal such as a brain wave or a neural current, or refers to an electrode used for extracting and measuring bio-information signals transmitted from inside the body to outside of the body.

An electrode pad may refer to a device electrically coupled to a bio-measurement device via a connector, and may include a plurality of terminals. For example, one of the terminals hereinafter referred to as a “first terminal”, to be connected to a bio-electrode from among the plurality of terminals transfer bio-information detected by the bio-electrode from the bio-electrode to the bio-measurement device. Also, another one of the terminals, hereinafter referred to as a “second terminal”, not connected to the bio-electrode from among the plurality of terminals, in an embodiment may maintain information required by the bio-measurement device. Throughout the application, “first terminal” and “second terminal” refer to terminals that provide one or more individual electrical connectors of the electrode pad. In another example, the electrode pad may maintain information by forming a pattern of a matrix in a portion of an area of the electrode pad. Such a pattern of a matrix includes a pattern based on the information to be stored.

Also, the bio-electrode device may be part of a type-based bio-measurement device configured to include a bio-electrode and an electrode pad, and the bio-electrode device may be designed to be attachable to and detachable from the bio-measurement device. For example, a bio-electrode device is type-based in that it is configured for specific types of use. More particularly, the bio-electrode device may maintain information locally to enable the maintained information to be transferred to the bio-measurement device when the bio-electrode device is fastened to the bio-measurement device. At this point, the bio-measurement device can configure itself based on the settings stored, based on the type of bio-electrode. For example, the bio-measurement device includes a bio-measurement processor that is operatively connected to the bio-electrode device, when the bio-electrode device is attached, and the bio-measurement processor may read the information from the bio-electrode device.

Further, the bio-measurement device may refer to a device that analyzes a state of a user through receiving an input of bio-information of the user received from the bio-electrode device fastened to the bio-measurement device via the connector. The bio-measurement device, as discussed above, uses a bio-measurement processor to perform these operations. For example, the bio-measurement device may measure a size or a shape of several portions of a living organism, and represent a state of the living organism numerically. In an embodiment, such a numerical representation includes data that represent the state of a user as discussed above, and includes metrics or representative numbers that characterize information received about a size or a shape of a portion of an organism received by a bio-electrode.

More particularly, the bio-measurement device may collect information required for measuring and analyzing bio-information although the user fails to perform an additional inputting. In an embodiment, the bio-measurement processor of the bio-measurement device automatically receives information from the bio-electrode device to which the bio-electrode device is fastened.

Therefore, the bio-electrode device may facilitate convenience of a user and miniaturization of a device by avoiding the need for an input device for inputting information. In embodiments, the bio-electrode device may maintain information in the bio-electrode device to enable the maintained information to be transferred to the bio-measurement device automatically when the bio-electrode device is fastened to the bio-measurement device. The bio-measurement processor reads the maintained information from the bio-electrode device, and uses it to process the bio-information.

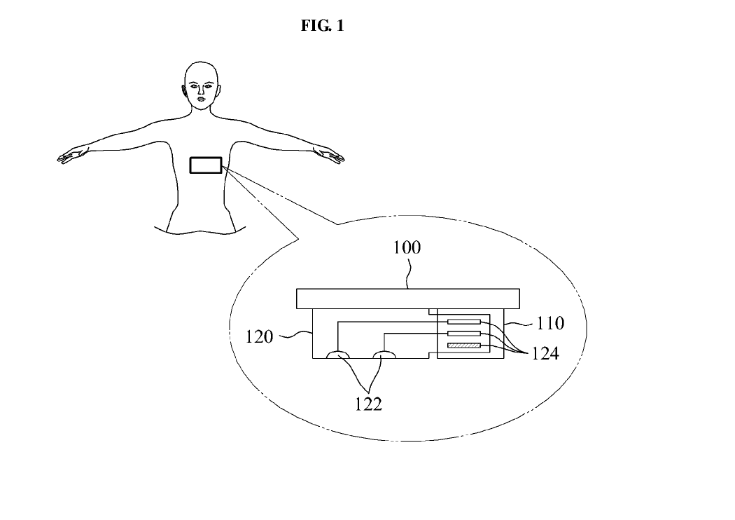

FIG. 1 illustrates an example of fastening a bio-electrode device 120 and a bio-measurement device 100.

Referring to FIG. 1, in embodiments the bio-measurement device 100 may be electrically connected to a connector 110, and may be connected to the bio-electrode device 120 through being fastened via the connector 110.

In an embodiment, the bio-measurement device 100 includes a bio-measurement processor. The bio-measurement processor is integrated into the bio-measurement device 100 in FIG. 1, but in other embodiments the bio-measurement processor may be separated from the bio-measurement device. The bio-measurement processor may be operatively connected to the bio-electrode device 120. The bio-measurement processor may be configured to read the information from the bio-electrode device and to otherwise provide information processing operations for the bio-measurement device 100.

In an embodiment, the bio-electrode device 120 includes a bio-electrode 122 for detecting bio-information of a user and an electrode pad 124, and is fastened to the connector 110 by an electrode pad 124. The bio-information is transferred to the bio-measurement device 100 via the connector 110 to be fastened through a first terminal of the electrode pad 124. The bio-measurement processor participates in processing the bio-information. More particularly, the bio-electrode device 120 maintains information in a portion of the electrode pad 124, and when fastened to the connector 110, allows the maintained information to be utilized for measuring and analyzing the bio-information performed in the bio-measurement device 100 through the maintained information being transferred to the bio-measurement device 100 along with the bio-information, for analysis by the bio-measurement processor. Examples of maintaining the information include using a second terminal and a matrix, details of which will be discussed later.

The bio-measurement device 100 may analyze a state of a user by receiving bio-information of the user from the bio-electrode device 120 fastened to the bio-measurement device 100 via the connector 110. Also, the bio-measurement device 100 may receive and read maintained information from the bio-electrode device 120 to utilize the maintained information in combination with the bio-information of the user for measuring and analyzing the state of the user. As discussed, such operations are performed by the bio-measurement processor.

The connector 110 refers to a device for fastening the bio-electrode device 120 when it is attached or detached from the bio-measurement device 100. In addition to facilitating the fastening, the connector 110 may transfer bio-information and other information of the bio-electrode device 120 to the bio-measurement device 100 for processing by the bio-measurement processor. In an example, the connector 110 includes a corresponding terminal for forming an electrical connection, for example, from the first terminal of the electrical pad 124 to transfer the bio-information. The connector 110 also includes a connecting part for providing access, to the bio-measurement device 100, to a second terminal or information maintained in a matrix. The second terminal includes a terminal not connected to the bio-electrode 122. The connector 110 may transfer these two types of information to the bio-measurement processor.

The bio-measurement device 100 fastened to the bio-electrode device 120 via the connector 110 provides consistent measurement and analysis of a physical state of a user by being attached to, for example, a body surface of the user.

By being constructed as discussed above, the bio-measurement device 100 facilitates user convenience and device miniaturization by receiving information by being fastened to the bio-electrode device 120 that maintains the information.

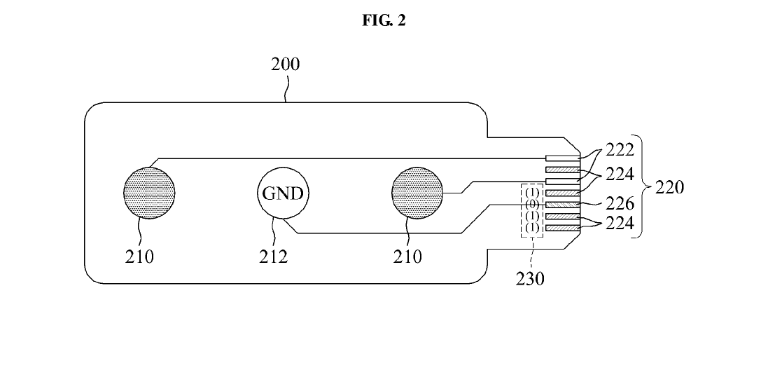

FIG. 2 illustrates an example of a detailed configuration of a bio-electrode device 200 that maintains information.

The bio-electrode device 200 of FIG. 2 may include bio-electrodes 210 and an electrode pad 220.

The bio-electrodes 210 may detect bio-information of a user through being attached to the user directly. In an example, the bio-electrodes 210 are placed in direct contact with the user, or alternatively the bio-electrodes are placed in contact with a conductive medium, such as a paste or gel, that allows the bio-electrodes 210 to detect and measure bio-information of a user. A form of the bio-electrodes 210 varies based on a type of the user. As an example, FIG. 2 illustrates the bio-electrodes 210 in a form of a circular patch reflecting a curvature of a body. By forming the bio-electrodes 210 with such a curvature, it is easier for the bio-electrodes 210 to maintain electrical contact with the body of the user, facilitating accurate measurement of bio-information of a user. In various aspects, the bio-electrodes 210 detect varied forms of data occurring in a body, for example, an electrocardiogram (ECG), a sound of heartbeat, and the like. However, the bio-electrodes 210 are not limited to measuring information about the cardiovascular system of the user, and may use electrical signals received by the bio-electrodes 210 to measure other types of information. For example, in some embodiments the bio-electrodes 210 gather information about characteristics of the nervous system of the user and/or measure brain activity, such as through electroencephalogram (EEG) data, or other nerve transmission signal data. Similarly, the bio-electrodes 210 also gather information about the activity of other systems or other aspects of the physiology of the user.

The electrode pad 220 may include a plurality of terminals. The plurality of terminals may be divided into a first terminal 222 and a second terminal 224, based on the connectivity of the terminals with the bio-electrodes 210. In an example, a first terminal 222 of the plurality of terminals is connected to the bio-electrodes 210, and the second terminal 224 is not connected to the bio-electrodes 210. In an embodiment, a terminal 226 connected to an electrode 212 in a state of being grounded is classified as belonging to the second terminal 224.

The first terminal 222 is electrically connected to the bio-electrodes 210. Because of the connection between the first terminal 222 and the bio-electrodes 210, the first terminal 222 performs a function of transferring bio-information detected by the bio-electrodes 210 to the bio-measurement device 100 when fastened to the connector 110, for processing by the bio-measurement processor.

In one embodiment, the second terminal 224 refers to a spare terminal not connected to the bio-electrodes 210. The bio-electrode device 200 maintains information, using the spare second terminal 224.

As used herein, maintained information includes property data associated with the bio-electrodes 210. Such property data describes factors such as a distance between neighboring bio-electrodes 210, impedance of the bio-electrodes 210, and the like, and include characteristics data associated with a user, such as a gender, a height, a weight, a body mass index (BMI), and the like. However, these examples should not be taken as limiting and other types of data similar to these examples are possible in other embodiments. The maintained information describes the configuration of the bio-electrodes 210, and/or the subject producing the data. As a result, the maintained information is useful in interpreting and processing the raw signals and information produced by the bio-electrodes 210.

In an illustrative example of the maintained data, the information refers to the property data associated with the bio-electrode device 200, for example, a number of bio-electrodes 210, a distance between the bio-electrodes 210, and the like. Accordingly, as the bio-electrode device 200 is connected through being fastened to the bio-measurement device 100 of FIG. 1, the property data may be measured via the bio-measurement device 100 at the bio-measurement processor by reading the property data from the bio-electrode device 200 automatically. By so doing, an inconvenience of separate input of the property data is avoided, and a measurement error may also be prevented.

If the bio-measurement processor of the bio-measurement device 100 reads the property data from the bio-electrode device 200 automatically, it may use the property data to interpret the bio-information produced by the bio-electrodes 210. For example, if the property data includes information about the distance between the bio-electrodes 210, the distance information may provide context that allows the bio-measurement processor to interpret the signals produced by the bio-electrodes 210, as given signal data would otherwise indicate different things based on how the bio-electrodes are situated.

As another example of using the maintained information, the maintained information may refer to characteristic data of the user, for example, a gender, a BMI, and the like, of the user. Accordingly, in these examples, the bio-measurement device 100 does not need to receive an input of the characteristics data directly from the user when it requires characteristics data. Instead, the bio-measurement device 100 may be provided with the characteristics data from the bio-electrode device 200 through being fastened to the bio-electrode device 200. Various embodiments provide multiple ways to manage the characteristic data of the user. In one approach, the user selects, as a product, the bio-electrode device 200 that maintains physiological information matched to a physiological property type of the user as the characteristics data through coding of the physiological information. In certain embodiments, the user may transfer the physiological information of the user to a producer of the bio-electrode device 200 through an Internet webpage, a call center service, and the like, and receives, as a product, a delivery of the bio-electrode device 200 that maintains the characteristics data through the physiological information being coded.

In the maintaining of the information, the electrode pad 220 maintains the information based on whether at least a portion of the second terminal 224 is connected to a ground. The connecting to the ground may refer to connecting a conducting wire to a ground region for a purpose of generating a zero potential. According to an example, the connecting to the ground is performed by connecting the electrode 212 in the state of being grounded from among the bio-electrodes 210 to a predetermined terminal 226 from among the terminals that make up the second terminal 224.

Referring to FIG. 2, the bio-electrode device 200 includes at least one bio-electrode 210, having a conductivity, for detecting bio-information. One of the bio-electrodes 210 refers to the electrode 212 in a grounded state. Also, the bio-electrode device 200 may include the electrode pad 220 in a portion that is designed to be fastened to the connector 110.

The electrode pad 220 includes the first terminal 222, designed to transfer the bio-information, the second terminal 224, which is not connected to the bio-electrodes 210, and the plurality of terminals.

The electrode pad 220 alternatively maintains information, using an array pattern of the terminal 226 connected to the electrode 212 in the grounded state, and the second terminal 224 not connected to the electrode 212 in the grounded state. For example, the electrode pad 220 of FIG. 2 may maintain binary information “1011” 230 by setting a digital value of “0” to correspond to the terminal 226 connected to the electrode 212 in the grounded state, and by setting a digital value of “1” to correspond to the second terminal 224 not connected to the electrode 212 in the grounded state.

The bio-measurement processor of the bio-measurement device 100 may be connected to the bio-electrode device 200 through being fastened via the connector 110, and may read the information maintained in the bio-electrode device 200, based on whether at least a portion of the second terminal 224 is connected to a ground. For example, in the configuration provided in FIG. 2, the bio-measurement device 100 reads the binary information “1011” 230 based on the interpretation of the grounded and ungrounded terminals discussed above.

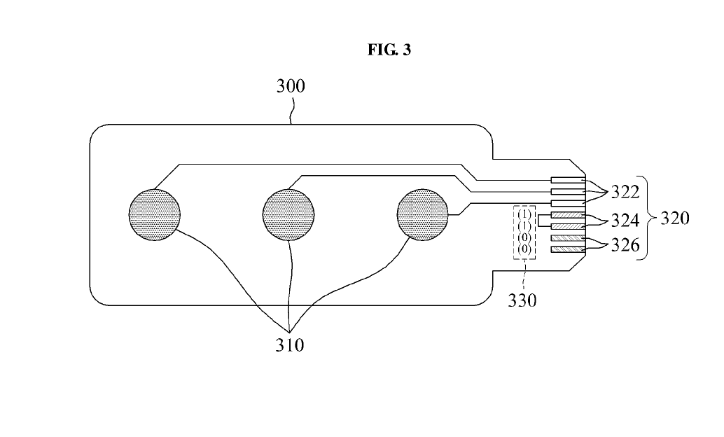

FIG. 3 illustrates another example of a detailed configuration of a bio-electrode device 300 that maintains information.

The bio-electrode device 300 includes bio-electrodes 310 and an electrode pad 320 including a plurality of terminals of a first terminal 322 connected to the bio-electrodes 310 and a second terminal not connected to the bio-electrodes 310. The bio-electrode device 300 maintains the information, using the second terminal.

When maintaining the information, the electrode pad 320 maintains the information based on whether at least a portion of the second terminal is connected to another terminal of the electrode pad 320. The connection to the other terminal may refer to electrically connecting a plurality of terminal pairs that are both identified as belonging to the second terminal.

Referring to FIG. 3, the bio-electrode device 300 may include at least one bio-electrode 310 having a conductivity for detecting bio-information. The bio-electrode device 300 may include the electrode pad 320 as a portion of the bio-electrode device 300 to be fastened to the connector 110.

The electrode pad 320 includes the first terminal 322 for transferring the bio-information and a plurality of second terminals not connected to the bio-electrodes 310.

In embodiments, the plurality of second terminals is divided into groups. In terminal group 324, a portion of the terminals the of second terminal of the electrode pad 320 are connected to one another. In another portion of the second terminals of the electrode pad 320, a terminal group 326 includes a portion of the terminals of the second terminal that are not connected to one another. Alternatively, the bio-electrode device 300 maintains information using an array pattern between the terminal groups 324 and 326, through identifying the terminal groups 324 and 326 that differ from one another based on whether their constituent terminals are connected to one another. In an example, the electrode pad 320 of FIG. 3 sets a digital value of “1” to correspond to a respective terminal of the terminal group 324 where the terminals are connected to one another, and sets a digital value of “0” to correspond to a respective terminal of the terminal group 326 where the terminals are not connected to one another, to maintain binary information “1100” 330.

The bio-measurement processor of the bio-measurement device 100 is connected to the bio-electrode device 300 through being fastened via the connector 110, and reads the information maintained in the bio-electrode device 300, based on whether each of the portions of the second terminal is connected to another terminal of the second terminal of the electrode pad 320.

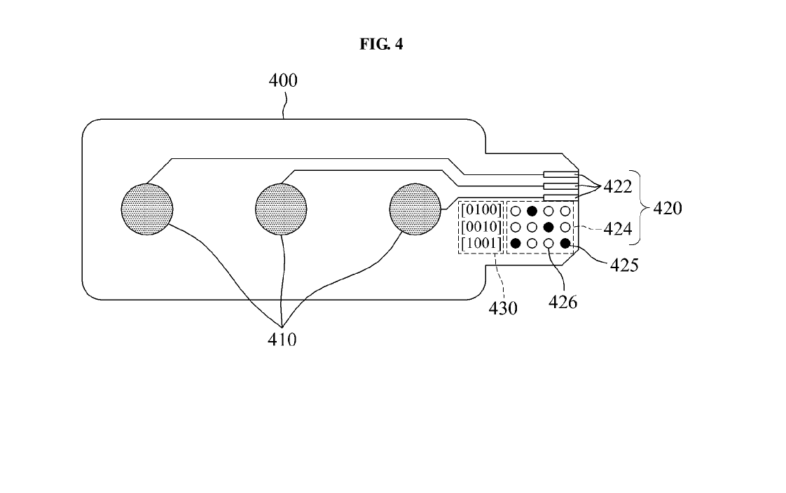

FIG. 4 illustrates an example of detailed configuration of a bio-electrode device 400 including a matrix that maintains information.

The bio-electrode device 400 of FIG. 4 includes bio-electrodes 410, an electrode pad 420, and a dot matrix 424. The bio-electrodes 410 detect bio-information of a user, through being attached to the user, possibly directly. The electrode pad 420 of FIG. 4 includes at least one terminal connected to the bio-electrodes 410. In an example, the electrode pad 420, configured to have a first terminal 422 electrically connected to the bio-electrodes 410, performs a function of transferring the bio-information detected from the bio-electrodes 410 to the bio-measurement device 100 when fastened to the connector 110.

The dot matrix 424 of FIG. 4 includes at least one of a conductive member 425 and an insulating member 426. The conductive member 425 may be composed of materials of which an electric conductivity is relatively high, and the insulating member 426 may be composed of materials having an insulating property, and therefore having a relatively low conductivity.

The dot matrix 424 of FIG. 4 maintains the information, using an array of the conductive member 425 and the insulating member 426. The dot matrix 424 may maintains the information, using a combination of binary information represented by the conductive member 425 and the insulating member 426, respectively, by disposing the conductive member 425 and the insulating member 425 in a form of a matrix, based on a predetermined rule. An example of the use of such a matrix is discussed, below.

Referring to FIG. 4, the bio-electrode device 400 includes at least one bio-electrode 410 having a conductivity for detecting the bio-information. The bio-electrode device 400 also includes the electrode pad 420 in a portion to be fastened to the connector 110.

The electrode pad 420 includes the first terminal 422 that transfers the bio-information, and includes the dot matrix 424 in at least a portion of an area of the electrode pad 420.

The dot matrix 424 may dispose the conductive member 425 and the insulating member 426 at predetermined positions in a two-dimensional array. In an example, the bio-electrode device 400 maintains the information, using an array pattern between the conductive member 425 and the insulating member 426, by identifying patterns of how the conductive member 425 and the insulating member 426 differ from one another throughout the array.

In an example, the dot matrix 424 of FIG. 4 sets a digital value of “1” to correspond to a portion of the array that is composed of the conductive member 425, and sets a digital value of “0” to correspond to a portion of the array that is composed of the insulating member 426, and maintain binary information “0100 0010 1001”. As shown in FIG. 4, some of the portions of the array are composed of the conductive member, and other portions are composed of the insulating member, and the bio-electrode device 400 uses the configuration of the array to encode binary information.

The bio-measurement processor of the bio-measurement device 100 is connected to the bio-electrode device 400, through being fastened via the connector 110, and reads the information maintained in the bio-electrode device 400, using an array of the conductive member 425 and the insulating member 426 as described above.

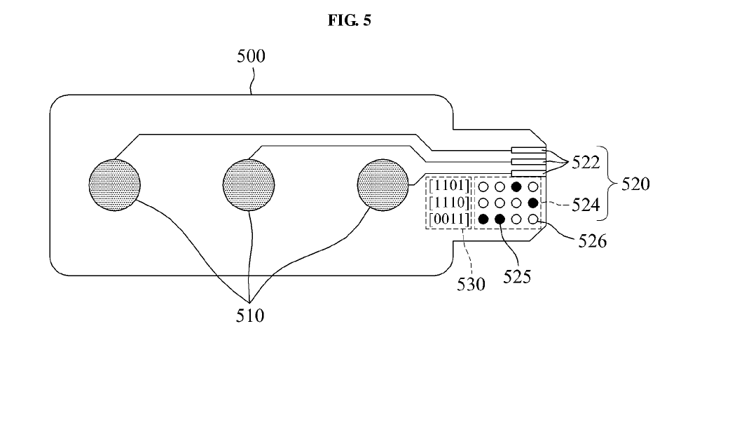

FIG. 5 illustrates another example of a detailed configuration of a bio-electrode device 500 including a matrix that maintains information.

The bio-electrode device 500 of FIG. 5 includes bio-electrodes 510, an electrode pad 520, and an optical dot matrix 524. Thus, FIG. 5 illustrates an embodiment that is similar to that of FIG. 4, except that instead of the dot matrix encoding information by using materials of different conductivities, the dot matrix uses dots with different optical properties.

In an embodiment, the bio-electrodes 510 are attached to a user directly, to detect bio-information of the user.

The electrode pad 520 includes at least one terminal connected to the bio-electrodes 510. In an example, the electrode pad 520, configured to have a first terminal 522 electrically connected to the bio-electrodes 510, performs a function of transferring the bio-information detected from the bio-electrodes 510 to the bio-measurement device 100 when fastened to the connector 110, where the bio-measurement processor receives it.

The optical dot matrix 524 may include at least one of a transparent member 525 and a non-transparent member 526. The transparent member 525 may be composed of a material permitting a passage of light when diffusion of light is present, and the non-transparent member 526 may be composed of material that failing to permit a passage or an entrance of light under the same conditions. Thus, such a dot matrix can be used to encode information because shining light upon the dot matrix produces a pattern of areas of the matrix through which light passes, and areas of the matrix through which light is blocked. Such a pattern of bright and dark spots may be processed to yield data based on the pattern.

Thus, the optical dot matrix 524 may maintain information, using an array of the transparent member 525 and the non-transparent member 526. When maintaining the information, the optical dot matrix 524 may maintain the information, using a combination of binary information represented by the transparent member 525 and the non-transparent member 526, respectively, through disposing the transparent member 525 and the non-transparent member 526 in a form of a matrix, based on a predetermined rule. Such a predetermined rule defines which portions of the matrix are associated with which values.

Referring to FIG. 5, the bio-electrode device 500 includes at least one bio-electrode 510 having a conductivity, for detecting bio-information. The bio-electrode device 500 may include the electrode pad 520 in a portion to be fastened to the connector 110.

The electrode pad 520 includes the first terminal 522 for transferring the bio-information, and includes the optical dot matrix 524 in at least a portion of an area.

The optical dot matrix 524 locates the array produced by the transparent member 525 and the non-transparent member 526 at a predetermined position. The bio-electrode device 500 thus maintains information, using an array pattern between the transparent member 525 and the non-transparent member 526, through identifying the transparent 525 and the non-transparent member 526 as having optical patterns that differ from one another. In an example, the optical dot matrix 524 of FIG. 5 sets a digital value of “1” to correspond to the transparent member 525, and sets a digital value of “0” to correspond to the non-transparent member 526, to maintain binary information “1101 1110 0011” 530.

The bio-measurement device 100 is connected to the bio-electrode device 500 through being fastened via the connector 110, and the bio-measurement processor of the bio-measurement device 100 reads the information maintained in the bio-electrode device 500, using an array of the transparent member 525 and the non-transparent member 526 as described above.

Accordingly, the bio-electrode device 120 may facilitate miniaturization of the bio-measurement device 100 and may enhance user convenience, through the information being transferred to the bio-measurement processor of the bio-measurement device 100 automatically when the bio-electrode device 120 is fastened to the connector 110, by electrically including a variety of useful information such as characteristics data associated with a user, property data associated with the bio-electrode 122, and the like, in the bio-electrode 122, through use of expendable supplies.

The information transferred to the bio-measurement device 100 in the bio-electrode device 120 through the fastening may be used for obtaining an appropriate biosignal based on a property of the bio-electrode 122, including property data of the bio-electrode 122, such as a distance between the bio-electrodes 122, impedance, and the like. Such information is encoded in the bio-electrode device 120 as provided in FIGS. 2-5, above, in various embodiments. The information may be interpreted by the bio-measurement processor of the bio-measurement device 100 in order to provide the bio-measurement device 100 with information about how to process and manage information derived from the bio-electrode device 120.

Example types of the information include characteristics data, such as a gender, a height, a weight, a BMI, and the like, of an object.

According to an example, health information of a user is transferred to the bio-measurement device 100 indirectly, through purchasing a type suitable for the user, from among product groups associated with a disposable bio-electrode device available for purchase, through being wrapped for a plurality of typical user groups. For example, the bio-measurement device 100 is coded for women within a certain height and weight range. Thus, a patient can purchase a measurement device that automatically indicates that it is to be used with a patient population and there is no need to individually code the measurement device for an individual user because the bio-measurement device 100 pre-codes attributes of the user, as discussed above.

According to an example use case, health information of a user is transferred to the bio-measurement device 100 indirectly, through inputting the health information of the user on an Internet webpage, and the like, and receiving a delivery of a product associated with a disposable bio-electrode device custom-made based on the inputted information. For example, a patient can input the information discussed above, such as height, weight, BMI, and so on, and embodiments provide a way to include in the bio-electrode device 120 information that automatically enables the bio-measurement device 100 to customize itself for a given patient with a minimum of effort.

Hereinafter, an operation of implementing the bio-electrode device 120 will be discussed in detail.

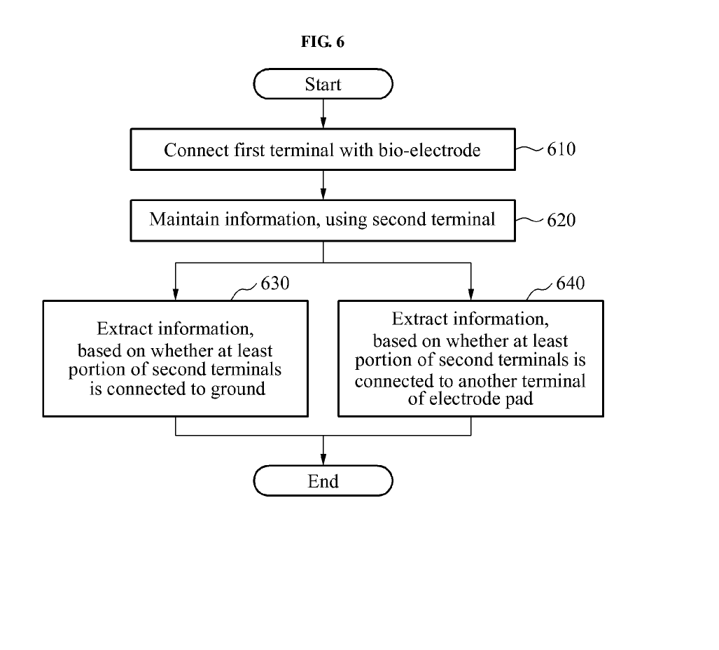

FIG. 6 illustrates an example of a method for implementing a bio-electrode device 120.

The method for implementing the bio-electrode device 120 may be performed in accordance with the description of the aforementioned bio-electrode device 120.

In 610, the bio-electrode device 120 connects at least one first terminal of the plurality of terminals of the electrode pad 110 with the bio-electrode 122. In 610, in an example, the first terminal is electrically connected to the bio-electrode 122, and the bio-information detected in the bio-electrode 122 is transferred to the bio-measurement device 100 when the electrode pad 110 is fastened to the connector 110.

In 620, the bio-electrode device 120 maintains information, using the second terminal not connected to the bio-electrode 122 from among the plurality of terminals of the electrode pad 110. In 620, in an example, the information is maintained, using the second terminal, being a spare terminal, not connected to the bio-electrode 122. Here, the information includes include property data associated with a distance between neighboring bio-electrodes 122, impedance of the bio-electrodes 122, and the like, and includes characteristics data associated with a gender, a height, a weight, a BMI, and the like, of a user. By maintaining this information using the second terminal, it is possible to produce a bio-electrode device that is pre-adapted to usage in certain situations.

After 620, the method may continue at 630 or 640, depending on the embodiment and the approach used in the embodiment used to maintain the information, using the second terminal. As will be discussed, 630 bases information storage on whether potions of the second terminal is connected to a ground. As will be discussed, 640 bases information storage on whether at least a portion of the second terminals is connected to another terminal of the electrode pad.

In 630, the bio-electrode device 120 extracts the information, based on whether at least a portion of the second terminals not connected to the bio-electrode 122 from among the plurality of terminals is connected to a ground. In 630, in an example, the information is extracted, using an array pattern of the second terminals connected to the electrode in a grounded state and the second terminals not connected to the electrode in the grounded state. As shown in FIG. 2, the bio-electrode device 120 may set a digital value of “0” to correspond to the terminal connected to the electrode in the grounded state, and may set a digital value of “1” to correspond to the second terminals not connected to the electrode in the grounded state, to read binary information “1011” in the bio-measurement device 100 by extracting the binary information “1011”. However, it is also possible to use the opposite approach, in which the bio-electrode device 120 sets a digital value of “1” to correspond to the terminal connected to the electrode in the grounded state, and set a digital value of “0” to correspond to the second terminals not connected to the electrode in the grounded state.

In 640, the bio-electrode device 120 extracts the information, based on whether at least a portion of the second terminal not connected to the bio-electrode 122 from among the plurality of terminals is connected to other terminals of the electrode pad 110. In 640, in an example, under a condition in which a terminal group, a portion of the second terminal, is connected to one another, and another terminal group, a portion of the second terminal, is not connected to one another, information is extracted, using an array pattern between the terminals groups by identifying the terminal groups that differ from one another. As shown in FIG. 3, the bio-electrode device 120 sets a digital value of “1” to correspond to a respective terminal of the terminal group connected to one another, and set a digital value of “0” to correspond to a respective terminal of the terminal group not connected to one another, to read binary information “1100” by the bio-measurement processor in the bio-measurement device 100 through extracting the binary information “1100”. However, it is also possible to use the opposite approach, in which the bio-electrode device 120 sets a digital value of “0” to correspond to a respective terminal of the terminal group connected to one another, and set a digital value of “1” to correspond to a respective terminal of the terminal group not connected to one another.

In another example, the bio-electrode device 120 sets the dot matrix 424 including at least one of the conductive member 425 and the insulating member 426 in a portion of an area of the electrode pad 110, and maintains the information, by the dot matrix 424, using an array of the conductive member 425 and the insulating member 426.

In this example, the electrode pad 110 includes the first terminal transferring bio-information, and includes the dot matrix 424 in at least a portion of an area. The dot matrix 424 may dispose the conductive member 425 and the insulating member 426 at predetermined positions, based on the information which is desired to be encoded.

Alternatively, the bio-electrode device 120 maintains information, using an array pattern between the conductive member 425 and the insulating member 426 through identifying the locations on the dot matrix 424 where conductive member 425 and the insulating member 426 are situated as differing from one another. As shown in FIG. 4, the bio-electrode device 120 sets a digital value of “1” to correspond to the conductive member 425 in the dot matrix 424, and set a digital value of “0” to correspond to the insulating member 426, to read binary information “0100 0010 1001” in the bio-measurement device 100, through extracting the binary information “0100 0010 1001”. As discussed above with respect to other embodiments, the significance of the “0” and the “1” may be switched.

In another example, the bio-electrode device 120 sets the optical dot matrix 524 including at least one of the transparent member 525 and the non-transparent member 526 in a portion of an area of the electrode pad 110, and maintains information, by the optical dot matrix 524, using an array of the transparent member 525 and the non-transparent member 526.

In this example, the electrode pad 110 includes the first terminal transferring bio-information, and may include the optical dot matrix 524 in at least a portion of an area. The optical dot matrix 524 disposes the transparent member 525 and the non-transparent member 526 at predetermined positions to encode information based on the disposition of the transparent member 525 and the non-transparent member 526.

In such an example embodiment, the bio-electrode device 120 maintains information, using an array pattern provided by the transparent member 525 and the non-transparent member 526, through identifying the transparent member 525 and the non-transparent member 526 differing from one another. As shown in FIG. 5, the bio-electrode device 120 sets a digital value of “1” to correspond to the correspond member 525 in the optical dot matrix 524, and sets digital value of “0” to correspond to the non-transparent member 526, to read binary information “1101 1110 0011” in the bio-measurement device 100 through extracting the binary information “1101 1110 0011”. As discussed above with respect to other embodiments, the significance of the “0” and the “1” may be switched.

According to an example use case, health information of a user is transferred to the bio-measurement device 100 indirectly, through purchasing a type of bio-electrode device suitable for the user from among product groups associated with a disposable bio-electrode device available for purchase, through being wrapped and prepared for a plurality of typical user groups. As discussed above, disposable bio-electrode devices are designed to be of different types that operate with different operating parameters and are designed for use with different patient populations. The bio-electrode devices 120 of embodiments are prepared so as to be customized for a particular operating scenario and patient parameters, and store information that describes their intended operating scenario and patient parameters to the bio-measurement device 100, for use by the bio-measurement processor, increasing convenience and simplicity.

According to an example, a bio-electrode device in which information including property data of a bio-electrode or characteristics data of a user is recorded may be provided in a plurality of types in stores through an appropriate packaging.

Accordingly, the user may use the bio-electrode device 120 including appropriate information through purchasing a type of packaging that reflects physiological health information and a type and a property of the bio-electrode device 120 to be used. Therefore, an issue of having to input information when replacing the bio-electrode device 120 is resolved through selecting a product once that was previously customized for its intended use.

The user may discuss, with a sales consultant, the physiological health information and the type and the property of the bio-electrode device 120 to be used. The sales consultant may select a type of the bio-electrode device corresponding to the information including the property data of the bio-electrode device 120 about which the user discussed with the sales consultant and the characteristics data of the user, and transfer the type of the bio-electrode device via a website, a delivery, and the like, through an appropriate packaging.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

The methods described above can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The media may also include, alone or in combination with the software program instructions, data files, data structures, and the like. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, WiFi, etc.). In addition, functional programs, codes, and code segments for accomplishing the example disclosed herein can be construed by programmers skilled in the art based on the flow diagrams and block diagrams of the figures and their corresponding descriptions as provided herein.

As a non-exhaustive illustration only, a terminal/device/unit described herein may be a mobile device, such as a cellular phone, a personal digital assistant (PDA), a digital camera, a portable game console, an MP3 player, a portable/personal multimedia player (PMP), a handheld e-book, a portable laptop PC, a global positioning system (GPS) navigation device, a tablet, a sensor, or a stationary device, such as a desktop PC, a high-definition television (HDTV), a DVD player, a Blue-ray player, a set-top box, a home appliance, or any other device known to one of ordinary skill in the art that is capable of wireless communication and/or network communication.

A computing system or a computer may include a microprocessor that is electrically connected to a bus, a user interface, and a memory controller, and may further include a flash memory device. The flash memory device may store N-bit data via the memory controller. The N-bit data may be data that has been processed and/or is to be processed by the microprocessor, and N may be an integer equal to or greater than 1. If the computing system or computer is a mobile device, a battery may be provided to supply power to operate the computing system or computer. It will be apparent to one of ordinary skill in the art that the computing system or computer may further include an application chipset, a camera image processor, a mobile Dynamic Random Access Memory (DRAM), and any other device known to one of ordinary skill in the art to be included in a computing system or computer. The memory controller and the flash memory device may constitute a solid-state drive or disk (SSD) that uses a non-volatile memory to store data.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A bio-electrode device, comprising:
a bio-electrode; and
an electrode pad including a plurality of terminals,
wherein a first terminal from among the plurality of terminals is connected to the bio-electrode, and
the electrode pad is configured to maintain information using a second terminal not connected to the bio-electrode, from among the plurality of terminals.

2. The bio-electrode device of claim 1, wherein the information is maintained based on whether at least a portion of the second terminal is connected to a ground.

3. The bio-electrode device of claim 1, wherein the information is maintained based on whether at least a portion of the second terminal is connected to another terminal of the electrode pad.

4. The bio-electrode device of claim 1, further comprising:
a dot matrix comprising at least one of a conductive member and an insulating member.

5. The bio-electrode device of claim 4, wherein the dot matrix is configured to maintain information using an array of the conductive member and the insulating member.

6. The bio-electrode device of claim 1, further comprising:
an optical dot matrix comprising at least one of a transparent member and a non-transparent member.

7. The bio-electrode device of claim 6, wherein the optical dot matrix is configured to maintain information using an array of the transparent member and the non-transparent member.

8. A bio-measurement device, comprising:
a bio-electrode device, comprising:
a bio-electrode; and
an electrode pad including a plurality of terminals,
wherein a first terminal from among the plurality of terminals is connected to the bio-electrode, and
the electrode pad is configured to maintain information using a second terminal not connected to the bio-electrode, from among the plurality of terminals; and
a bio-measurement processor operatively connected to the bio-electrode device and configured to read the information from the bio-electrode device.

9. The bio-measurement device of claim 8, wherein the bio-measurement processor is configured to read information from the bio-electrode based on whether at least a portion of the second terminal, from among the plurality of terminals, is connected to a ground.

10. The bio-measurement device of claim 8, wherein the bio-measurement processor is configured to read information from the bio-electrode based on whether at least a portion of the second terminal, from among the plurality of terminals, is connected to another terminal of the electrode pad.

11. The bio-measurement device of claim 8, wherein bio-electrode device comprises a dot matrix comprising an array of a conductive member and an insulating member, and the bio-measurement processor is configured to read information from the bio-electrode based on the array of the conductive member and the insulating member.

12. The bio-measurement device of claim 8, wherein the bio-electrode device comprises an optical dot matrix including an array of a transparent member and a non-transparent member, and the bio-measurement processor is configured to read information from the bio-electrode based on the array of the transparent member and the non-transparent member.

13. The bio-electrode device of claim 1, wherein a first terminal of the plurality of terminals is connected to the bio-electrode, and at least a portion of a second terminal, not connected to the bio-electrode, from among the plurality of terminals, is connected to a ground.

14. A bio-electrode device, comprising:
a bio-electrode; and
an electrode pad including a plurality of terminals,
wherein a first terminal of the plurality of terminals is connected to the bio-electrode, and
at least a portion of at least one second terminal not connected to the bio-electrode, from among the plurality of terminals, is connected to another terminal of the electrode pad.

15. A method for implementing a bio-electrode device, comprising:
connecting a first terminal of a plurality of terminals of an electrode pad with a bio-electrode; and
maintaining information using a second terminal not connected to the bio-electrode, from among the plurality of terminals of the electrode pad.

16. The method of claim 15, further comprising:
extracting the information based on whether at least a portion of the second terminal is connected to a ground.

17. The method of claim 15, further comprising:
extracting the information based on whether at least a portion of the second terminal is connected to another terminal of the electrode pad.

18. The method of claim 15, further comprising:
connecting a terminal of a plurality of terminals of an electrode pad with a bio-electrode;
setting a dot matrix including a conductive member and an insulating member to be a portion of an area of the electrode pad; and
maintaining information, by the dot matrix, using an array of the conductive member and the insulating member.

19. The method of claim 15, further comprising:
connecting a terminal of a plurality of terminals of an electrode pad with a bio-electrode;
setting an optical dot matrix including a transparent member and a non-transparent member to be a portion of an area of the electrode pad; and
maintaining information, by the optical dot matrix, using an array of the transparent member and the non-transparent member.

20. A method of producing a customized bio-measurement device, comprising:
receiving a selection of configuration information for a bio-electrode device, the bio-electrode device comprising a bio-electrode and first terminal connected to the bio-electrode and a second terminal not connected to the bio-electrode;
configuring a portion of the bio-electrode device to store the configuration information; and
forming an operative connection between the bio-electrode device and a bio-measurement processor, wherein the operative connection allows the bio-measurement processor to read the configuration information.

21. The method of claim 20, wherein the portion of the bio-electrode device is the second terminal and the bio-electrode device is configured to store the configuration information by connecting at least a portion of a second terminal to a ground.

22. The method of claim 20, wherein the portion of the bio-electrode device is the second terminal and the bio-electrode device is configured to store the configuration information by connecting at least a portion of a second terminal to another terminal of the electrode pad.

23. The method of claim 20, wherein the portion of the bio-electrode device is configured to store the configuration information by using a dot matrix, using an array of a conductive member and an insulating member.

24. The method of claim 20, wherein the portion of the bio-electrode device is configured to store the configuration information by using an optical dot matrix, using an array of a transparent member and a non-transparent member
